(19) United States
(12) Patent Application Publication
(10) Pub. No.: US 2010/0228248 A1
(43) Pub. Date: Sep. 9, 2010

(54) Method and device for treating cancer with electrical therapy in conjunction with a catheter and high power pulser

(76) Inventor: Adam H. Griffin, San Diego, CA (US)

Correspondence Address: Adam H. GRIFFIN, 12097 Avenida Sivrita, San Diego, CA, 92128, US

(21) Appl. No.: 12/380,852

(22) Filed: Mar. 3, 2009

(51) Int. Cl. A61B 18/14

(52) U.S. Cl. 606/41

(57) ABSTRACT

Apparatus for delivering high power, short duration, low energy electric pulses associated with generating high electric fields across cancerous tumors. The apparatus has a small diameter catheter with conducting core and insulating sheath introduced through the femoral artery, orifice or incision with its tip routed to the treatment site. A counter electrode of generally round or flat shape introduced through another orifice, incision or artery sandwiches the tumor between the two electrodes. A high power electric pulser delivers a multiplicity of high voltage pulses to the treatment site producing a therapeutic effect or annihilation of cancer.

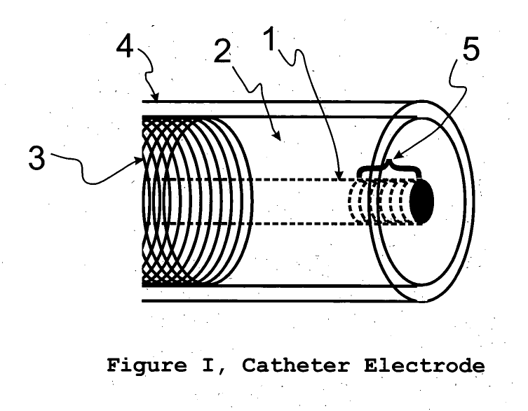

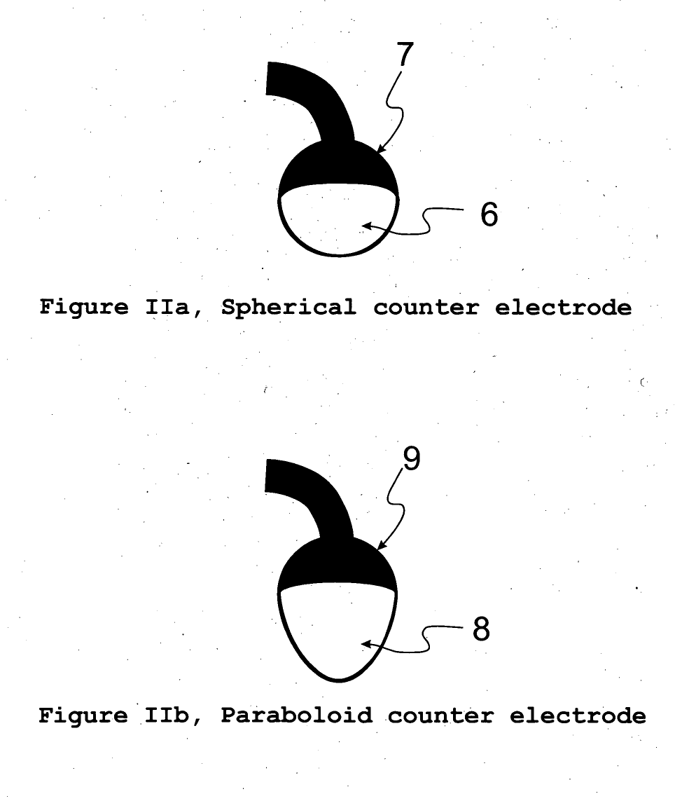

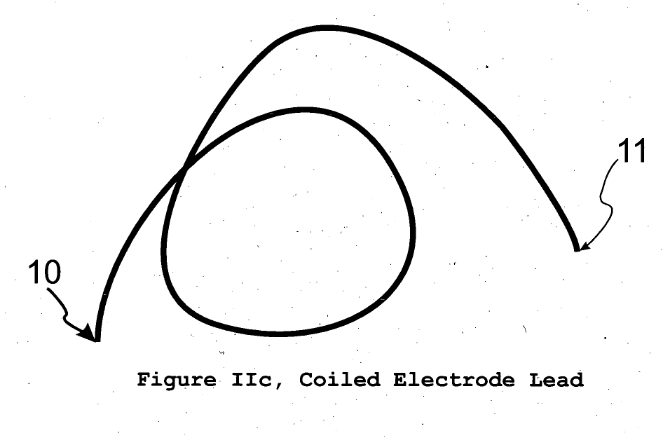

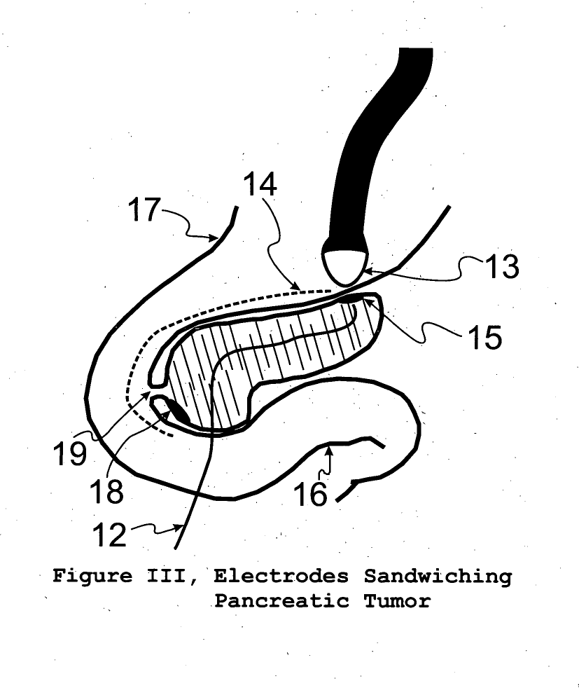

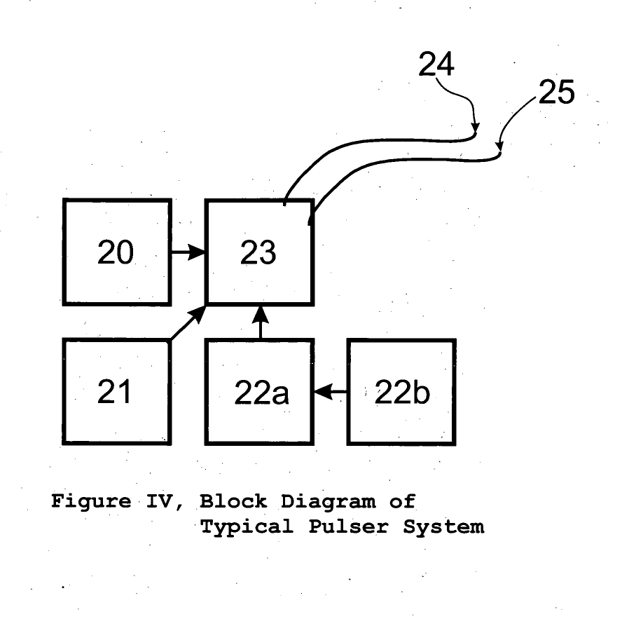

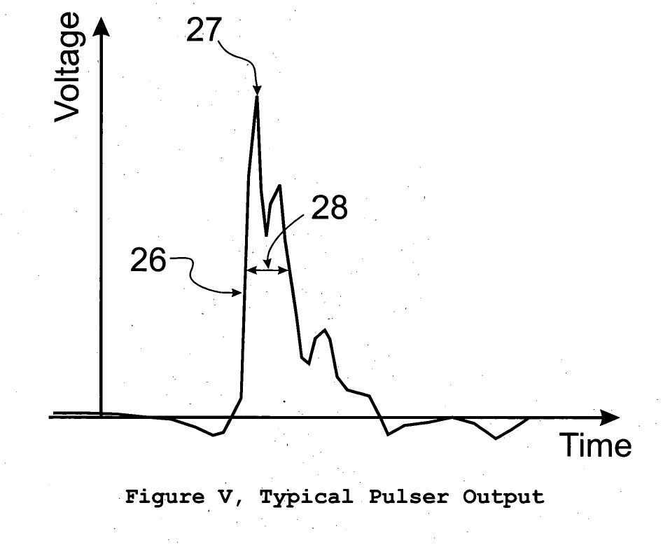

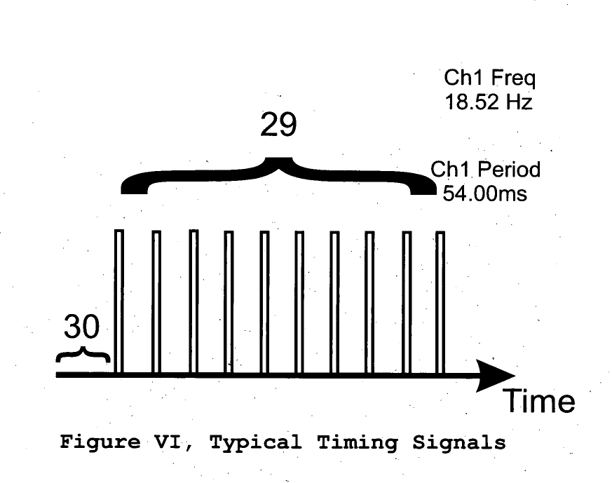

### BACKGROUND OF THE INVENTION

This invention relates to apparatus for cancer electrotherapy whereby cells are destroyed by high power and low energy pulsed electric fields delivered using a catheter and counter electrode from a pulsed power source.

At the 2006 Power Modulator Conference on May 14-18, 2006 researchers from Old Dominion University in New York presented a live mouse study (Nuccitelli, Schoenbach et. Al), “Nanosecond pulsed electric fields cause melanomas to self-destruct”, Biochemical and Biophysical Research Communications, Elsevier YBBRC 15884, 8 Mar. 2006.

Similar work on a live human was conducted at USC in Los Angeles (Garon, Gundersen et. Al), “In vitro and in vivo evaluation and a case report of intense nanosecond pulsed electric field as a local therapy for human malignancies”, Int. J. Cancer, Wiley Ed. Ref. No.: 06-1783R2 Date 19 Mar. 2007.

The present invention is based on the realization that effective cancer treatment can be effected by utilization of fast rise time, high voltage and short duration pulses delivered to the tumor site inside the human body using a catheter and counter electrode. Unlike the prior art affecting only the surface of skin using relatively large needle-like electrodes my invention allows for drug-free cancer treatment by employing a pulser means to actuate a sequence of impulses between two electrodes sandwiching a cancerous tumor. Also unlike prior art, my catheter tip is so small that electric fields are confined specifically to an area of interest within the human body. Cancer is however an insidious disease so the disappearance of inhibitors generated by one tumor becoming destroyed, can lead to increased growth of a different tumor.

### SUMMARY OF THE INVENTION

The object of this invention is to provide a new and relatively simple modality of cancer treatment for use by a doctor of internal medicine. Due to its small diameter catheter containing a center electrode and insulating sheath that can be introduced through the femoral artery and guided to the lesion or tumor it serves twofold purpose: (i) by sandwiching the tumor between the catheter tip or distal end and counter electrode a circuit comprising both electrodes is connected to a high power, low energy pulsed power source generating fast-rising, high voltage pulses and ensuing high electric field dependent on the shape and proximity of the electrodes, (ii) the invention invokes destruction of cancerous cells and tumors using no drugs.

The present invention is designed to break capillaries hence cutting off the blood supply to a tumor or causing cells to self-destruct. The present invention is based on the objective to treat cancerous tumors including within the pancreas, and allows specific delivery of pulsed electric fields; which may have additional therapeutic or stimulating effects for treating diabetes and other ailments.

### BRIEF DESCRIPTION OF THE DRAWINGS

Figure I shows one embodiment of a small diameter (e.g. 0.9 mm or 7 French) catheter electrode with e.g. 15 or 24 mil embedded electrode, radio opaque marker tip and steel braid.

Figure IIa shows one embodiment of a spherical counter electrode used in conjunction with the catheter electrode, and Figure IIb shows a second embodiment of a paraboloid counter electrode used in conjunction with the catheter electrode. Figure IIc demonstrates the electrodes of Figure I and II can be coiled.

Figure III shows an exemplary use of the invention to treat pancreatic cancer where the pancreas is predisposed between the stomach and small intestine. A small diameter catheter is introduced into the pancreas by means known to those skilled in the art of internal medicine. An alternative large surface area counter electrode is also shown unfurled around the pancreas, or using the counter electrode of Figure II.

Figure IV shows a typical system block diagram including electrodes and controls.

Figure V shows a typical pulsed voltage produced by the pulser of Figure IV.

Figure VI is a typical therapeutic timing signal produced by the system of Figure IV.

### DETAILED DESCRIPTION OF THE PREFERRED EMBODIMENT

Evidence that electric pulses of short duration and high power kills cancer is now available for a mouse and a human because of visionaries like Schoenbach at Old Dominion and Gundersen at USC. The work conducted by these authors concentrates on skin surface treatment of cancerous cells thus having proved the principle that pulsed electric fields are effective in destroying cancer cells through various processes including breaking of capillary blood supplies to tumors, and altering the cell membrane or interior geometry thus signaling macrophages to destroy these cells. Authors in this field have failed to convey a preferred method to eradicate or treat cancer is from the inside of a human body and with a small diameter catheter for example 0.9 mm diameter (7 French). The beauty of this type of short-pulse electrotherapy is that heat is not used to affect treatment nor is high energy required.

Electrotherapy of the type described by this invention employs high power, short duration pulses e.g. with rise times in the range 0.1 ns to 100 ns and pulse widths in the range 1 ns to 1000 ns. The electric field intensity impressed across a cancerous lesion or group of cells in the body is determined in part by the shape of the electrodes. In prior publications, what is claimed to be a catheter is unlike what is being disclosed herein and is simply a coaxial cable with center electrode and outer braid whereby needles are poked into the surface to cause electric field to penetrate the tissue and flow from center electrode to the braid via needles (the current then could flow laterally across the surface rather than normal to the tissue surface).

In this invention a small diameter catheter is introduced into the body using methods known to medical professionals similar to angioplasty and stenting of arteries. The catheter contains a conducting wire or media coated by an insulator. The process of making the catheter consists of starting with a mandrel wire and extruding insulating materials around that wire and possibly braiding to improve torquability of the wire. The braid would not necessarily extend all the way to the tip in order to avoid affecting the electric field profile at the tip.

A counter electrode or current return path is necessary to complete the circuit and is made using a conductive media. In this invention the catheter electrode and the counter electrode are connected to an appropriate pulsed electric field source or pulser. Referring to Figure I, the exposed tip (5) or distal end of a small diameter (e.g. 0.9 mm) catheter is composed of biocompatible Platinum with tip of diameter e.g. 15 mils or 24 mils. The insulator (2) is made from but not limited to materials including lubriciously coated layers of Pebax, Nylon, Polyimide, Vectra, FEP and PTFE. Such insulating layers are able to withstand 1000V per mil, or a 6 mil catheter wall thickness layer can withstand in excess of 10 kV. The catheter tip (5) is made from a radio opaque and biocompatible conductor such as Platinum or Platinum-Iridium and attached electrically to center conductor (1). Pushability and torquability of the catheter is improved by steel braid (3) disposed on insulator (2) at pre-determined distance from the catheter tip. The entire catheter structure of Figure I is overcoated with lubricious layer (4) implemented using e.g. Pebax containing Medglide brand or using e.g. treatment of Nylon by a company named Surmodics.

With the introduction of a counter electrode (Figure II) in close proximity to the catheter tip and use of an appropriate pulse source (Figure IV), an electric field intensity is created high enough to destroy cancerous cells with minimal invasiveness and without drugs. In this example of the use of this invention and having a look at the pancreas and geometry of surrounding tissue of Figure III the counter-electrode and electrodes shape the pulsed electric field whereby treatment is focused at the tip of the catheter. No heat is generated due to the short pulse duration and relatively slow pulse repetition rate. The desired effect is from a strong electric field (units of Volts per centimeter) impressed between the catheter tip and the counter electrode through the small intestine or stomach walls.

Figure IIa is a spherical counter electrode with exposed front surface (6) made from a biocompatible conductor such as Titanium or Platinum and a dielectric insulating layer (7). The tip of the counter electrode can also have a very thin layer of protective dielectric overcoat. Dielectric layer properties on the electrode surface can be adjusted to affect the shape or localized magnitude of the pulsed electric field. Figure IIb is a similar counter electrode but of paraboloid shape used to further modify the shape of the pulsed electric field with exposed front surface (8) and dielectric insulating layer (9). Each of the electrode and counter electrode structures are elongated and can be coiled as shown in Figure IIc where the proximal end (10) is connected to the pulser and the distal end (11) is inserted into the patient.

Figure III shows how the distal end of catheter electrode (12) of Figure I can be disposed with respect to the distal end of counter electrode (13) of Figure II. Note that for ease of discussion, not all elements of Figure III are drawn to scale. Figure III also suggests a third broad-area surface contour electrode (14) inserted into the body; this particular mode of treatment lends itself to pancreatic cancer therapy because a counter electrode formed by a flat sheet of conductive mesh or material having been rolled-up and then un-furled after insertion, can be made to encompass substantially around the top and bottom of the pancreas. This type of flattened, large surface area counter electrode (14) can be formed from a mesh of polypropylene with Carbon or other conductive material embedded so it is conductive yet biocompatible; for example the mesh used as a bladder sling could be modified for this purpose. The small diameter catheter (12) of Figure III is shown routed presumably from the Femoral Artery, into the Great Pancreatic Artery and finally to the point of the lesion or pancreatic tumor (15) to be treated.

An embodiment of the counter electrode of Figure II (round or parabolic shape) inserted through a small incision in the body and disposed adjacent to the catheter through the tissue is shown in Figure III. The counter electrode is thus disposed on the opposite side of stomach tissue (17) of Figure III with respect to the catheter electrode (12) distal tip and therefore a pulsed electric field generated is impressed across the lesion (15) providing therapeutic effects. The reader can also appreciate the counter electrode could be introduced into the body through a small incision and placed opposed to the tip of the catheter electrode (12). Alternatively for example, a tumor or lesion (18) can be treated similarly at a different site on the pancreas adjacent to small intestine (16). The reader will also recognize that catheter (12) or a suitable counter electrode (13) or a suitable counter electrode (14) could be introduced through the pancreatic duct (19) rather than being introduced e.g. through a network of arteries, orifices or incisions.

The process of fabricating the spherical or paraboloid counter electrode would be similar to other predicate devices familiar to those skilled in the art of internal medicine probes. Platinum tipped electrodes are known to be biocompatible and have been used for pacemaker leads. Titanium or steel would also be an acceptable material to form electrodes. Silicone is an acceptable coating or dielectric material as are other medical grade polymers including Pebax, Nylon and parylene.

Figure IV shows a block diagram of a typical pulser circuit used to generate high voltage impulses across the electrodes and therefore pulsed electric fields according to this invention, across the tissue or tumor. In Figure IV a power supply (20), high voltage source (21) and control circuit (22a) are driving the pulser (23). The control circuit (22a) is attached to the user interface (22b); typically a computer display or hardware including buttons, keys and knobs. The output of pulser (23) is in turn connected to catheter electrode (24) and counter electrode (25). The pulser of Figure IV is comprised of off-the-shelf components including switches, spark gaps, semiconductors, wiring, magnetic transformers, capacitors, logic circuitry, etcetera. Control of the pulser circuit can be implemented by well known means including microcontrollers and digital logic. Sensors and antennas can be incorporated into the pulser system.

The best mode contemplated in carrying out the invention is using the catheter of Figure I with Platinum tip (5) delivered via the femoral artery preceeded by a guide catheter and Xray contrast fluid well known to those skilled in the art; the Platinum tip not only providing electrical stimulus across the tumor but serving as a radio-opaque marker for catheter guiding by a doctor of internal medicine. The best mode for introducing a counter electrode would be through an orifice not limited to the larynx and consisting of a Platinum or e.g. Titanium tip also guided within close proximity of the catheter electrode.

Once the two electrodes are determined to be in close proximity the best mode contemplated is to deliver voltage pulses using the pulser of Figure IV and pulsed waveform similar to that of Figure V with fast rise of e.g. 15 nanoseconds (26), peak voltage e.g. 8 kV (27), and e.g. pulse width 100 nanoseconds (28) all of which are variables tunable to achieve the most efficacy. The intensity of the electric field generated across the tumor depends on the geometry and spacing of electrodes of Figures I and II with tissue sandwiched in between referring to the representative example of Figure III.

Sequences of pulses shown in Figure V are contemplated with timing as shown in Figure VI where ten pulses (29) occur at a repetition rate of approximately 18.5 Hz and separated by delay time (30) of typically 5 seconds between bursts, for a total of approximately 100 pulses per treatment. Not only should the reader understand that the pulse timing are variable to achieve the most efficacy, and that the timing pulses are necessarily of very much longer duration than the treatment impulse duration (28) to avoid tissue heating; where just one treatment pulse typically occurs at the rising edge of each timing pulse.

This invention can be used to treat other types of cancer for example colon, limb, brain etc. hence the scope of use for this invention is not limited to the pancreas whereby the example of Figure III is provided only to demonstrate the utility of this invention.

What I claim as new and desire to protect by Letters of Patent of the United States is:

1. An apparatus for sandwiching a cancerous tumor between two electrodes, said electrodes comprising an electrical circuit including:
small diameter catheter electrode introduced through the femoral artery consists of a conducting inner core and insulating, lubriciously coated outer core;
counter electrode introduced through an orifice or incision to the body is placed in proximity to the small diameter catheter electrode; and
means for actuating high power impulse signals between the two electrodes whereby a high power pulsed electric filed is impressed across the gap containing human tissue.

2. The apparatus according to claim 1, wherein said pulser actuating means includes for generating said impulse signals.

3. The apparatus according to claim 1, wherein said catheter and/or counter electrode includes for insertion into an orifice or incision in the human body.

4. The apparatus according to claim 1, wherein said catheter and/or counter electrode includes a radio opaque marker tip.

5. The apparatus according to claim 1, wherein said catheter and/or counter electrode includes a lubricious coating.
